(19) United States
(12) Patent Application Publication
(10) Pub. No.: US 2005/0015053 A1
(43) Pub. Date: Jan. 20, 2005

(54) Safety catheter

(75) Inventor: Jerry W. Parker, Costa Mesa, CA (US)

Correspondence Address: Troxell Law Office PLLC;Suite 1404, 5205 Leesburg Pike, Falls Church, VA, 22041, US

(73) Assignee: BakTech Corp.

(21) Appl. No.: 10/885,090

(22) Filed: Jul. 7, 2004

Related U.S. Application Data
(60) Provisional application No. 60/487,298, filed on Jul. 16, 2003.

(51) Int. Cl. A61M 5/178

(52) U.S. Cl. 604/164.12

(57) ABSTRACT

A safety catheter has a stand, a spring, a barrel, a hub, a cap and a seal. The stand and the spring are mounted slidably within the barrel. The stand has a proximal end, a distal end, an outer surface, a needle protruding from the proximal end and multiple annular notches formed in and extending radially inward from the outer surface. The barrel has a closed end, an open end, a hole formed in the closed end, and multiple clipper springs connected in the closed end and arranged around the hole. The clipper springs can clip the annular notches to hold the needle introduced into the hub and the needle with the hub is inserted into a patient. After insertion of the catheter is complete, the hub can be simply withdrawn from the catheter to let the needle retract in the barrel automatically to prevent accidental pricking of staff.

### BACKGROUND OF THE INVENTION

1. Field of the Invention

The present invention relates to a safety catheter, and more particularly to a safety catheter that withdraws an internal needle automatically.

2. Description of Related Art

A catheter is an instrument that is used to introduce certain fluids such as a saline solution directly into the bloodstream of a patient. Typically, a needle is first introduced through a cannula portion of the catheter and then into the skin of the patient at the desired location of the patient. Once insertion is complete, the needle is removed from the cannula portion of the catheter. After removing the needle, a fluid handling device such as a syringe is attached to the cannula. Fluid is then infused directly from the fluid handling device through the cannula of the catheter into the bloodstream of the patient.

When the needle is removed from the cannula, the medical personnel must place the exposed needle at a nearby disposal unit while simultaneously addressing the task of infusing the fluids into the patient. At this juncture, the exposed needle creates a danger of an accidental needle stick occurring which leaves the medical personnel vulnerable to the transmission of various, dangerous blood-borne pathogens. Careless disposal of used needles can put other health care workers at risk as well. Even others outside the health care profession, for example those involved in the clean-up and final disposal of medical waste can be at risk of an accidental needle stick from a carelessly discarded needle.

### SUMMARY OF THE INVENTION

The main objective of the present invention is to provide a safety catheter that withdraws an internal needle automatically to prevent accidental pricking of staff etc.

To achieve the objective, a safety catheter in accordance with the present invention comprises a stand, a spring, a barrel, a hub, a cap and a seal. The stand and the spring are mounted slidably within the barrel. The stand has a proximal end, a distal end, an outer surface, a needle protruding from the proximal end and multiple annular notches formed in and extended radially inward from the outer surface. The barrel has a closed end, an open end, a hole formed in the closed end, and multiple clipper springs connected in the closed end and around the hole. The clipper springs can clip the annular notches to hold the needle introduced into the hub and the needle with the hub is inserted into a patient. After insertion of the catheter is complete, the hub is simply withdrawn from the catheter to let the needle retract in the barrel automatically to prevent accidental pricking of staff etc.

Further benefits and advantages of the present invention will become apparent after a careful reading of the detailed description with appropriate reference to the accompanying drawings.

### BRIEF DESCRIPTION OF THE DRAWINGS

FIG. 1 is a partial exploded perspective view of a safety catheter in accordance with the present invention;

FIG. 2 is an exploded perspective view of the safety catheter in FIG. 1;

FIG. 3 is a cross sectional side view of the safety catheter in FIG. 1; and

FIG. 4 is an operational cross sectional side view of the safety catheter in FIG. 1 showing the catheter with a needle automatically retracted into a barrel.

### DETAILED DESCRIPTION OF THE INVENTION

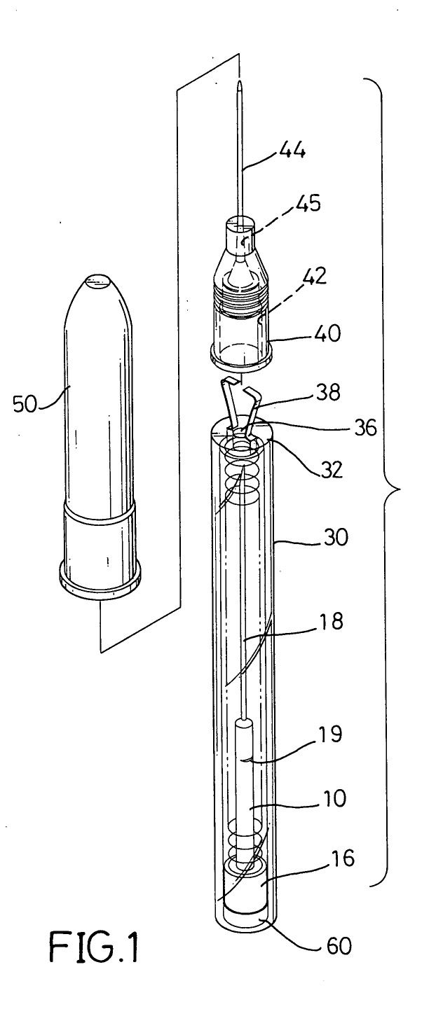

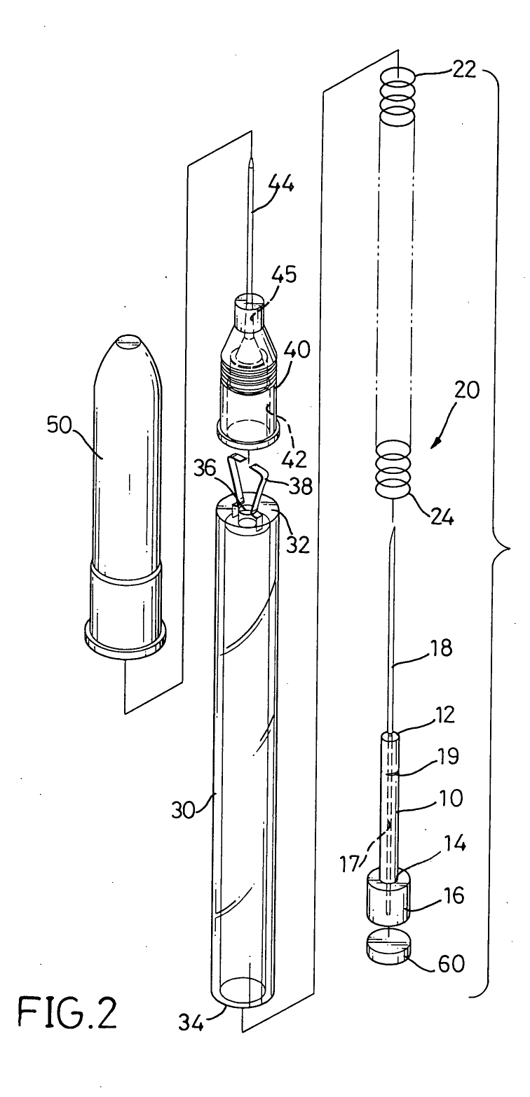

With reference to FIGS. 1 and 2, a safety catheter in accordance with the present invention comprises a stand (10), a spring (20), a barrel (30), a hub (40), an optional cap (50) and an optional seal (60).

The stand (10) is cylindrical, is mounted slidably in the barrel (30) and has a proximal end (12), a distal end (14), an axis, a head (16), a channel (17), a needle (18), an outer surface and multiple annular notches (19). The head (16) is formed on and extends longitudinally outward from the distal end (14). The channel (17) is formed along the axis. The needle (18) has a sharp end (not numbered) and a blunt end (not numbered). The blunt end of the needle (18) is inserted into the channel (17) and the sharp end protrudes from the proximal end. The multiple annular notches (19) are formed in and extend radially inward from the outer surface.

The barrel (30) has a closed end (32), an open end (34), a hole (36) and multiple clipper springs (38). The hole (36) is formed in the closed end (32). The multiple clipper springs (38) are connected in the closed end (32) and around the hole (36). Preferably, the clipper springs (38) are formed in pairs and each pair resembles a pair of tongs.

The spring (20) is mounted slidably in the barrel (30) and around the stand (10) and the needle (18) and has a proximal end (22) and a distal end (24). The distal end (24) abuts the head (16) of the stand (10) and the proximal end (22) abuts the closed end (32) of the barrel (30).

The hub (40) is mounted with the barrel (30) and has a proximal open end, a distal closed end, a chamber (42), a center channel (45) and a cannula (44). The chamber (42) is formed in the proximal open end. The center channel (45) is formed longitudinally and extends in the distal closed end to communicate with the chamber (42). The clipper springs (38) are compressed in the chamber (42) and let the hub (40) connect with the barrel (30). The cannula (44) is connected with the distal closed end and communicates with the channel (45).

The optional cap (50) covers the hub (40).

The seal (60) is mounted in the open end (34) of the barrel (30). The head (16) of the stand (10) abuts the seal (60) and the needle (18) is retained in the barrel (30) before use. Preferably, the seal (60) is a gas permeable seal.

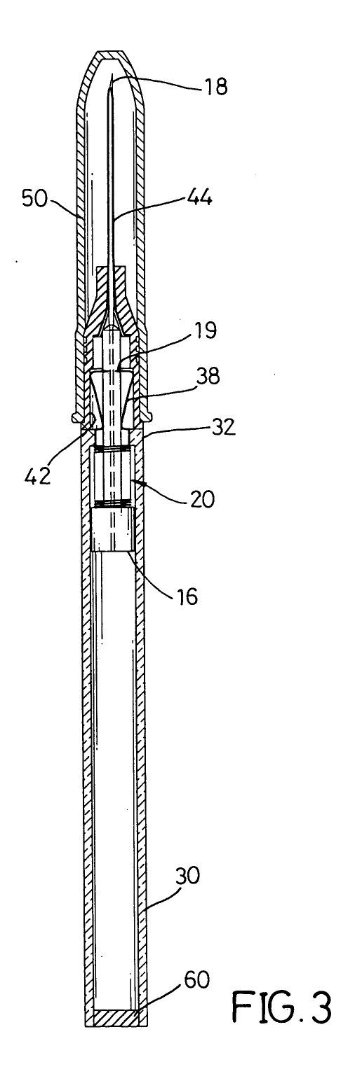

With further reference to FIG. 3, the head (16) is pushed toward the closed end (32) of the barrel (30) and compresses the spring (20). The proximal end (12) of the stand (10) and the needle (18) are pushed through the hole (36) of the barrel (30) until the clipper springs (38) clip the notches (19) in the stand (10). When the stand (10) is held by the clipper springs (38), the needle (18) passes through the cannula (44) and protrudes from the cannula (44).

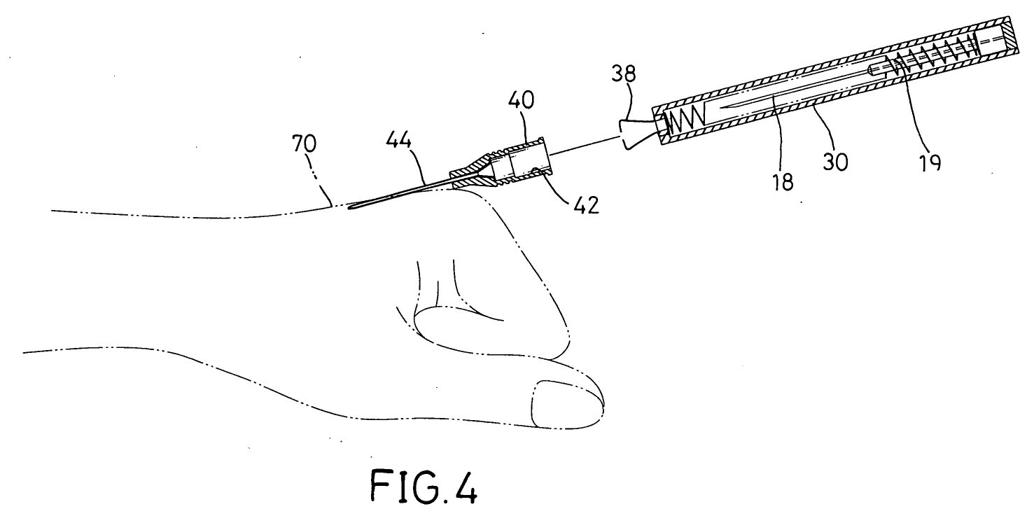

With further reference to FIG. 4, the safety catheter is used to insert into a part of a body (70). The needle (18) with the cannula (44) is inserted into the body (70). The barrel (30) pulls back to separate from the hub (40). Then, the hub (40) will stay in the body (70) for further use so the hub (40) simply separates from the clipper springs (38) of the barrel (30). When the clipper springs (38) leave the chamber (42) of the hub (40), the clipper springs (38) spring outward and let the notches (19) leave the clipper springs (38). Then, the stand (10) with the needle (18) is retracted in the barrel (30) by the tension of the spring (20).

The safety catheter in accordance with the present invention can retract the needle in the barrel automatically to prevent accidental pricking of staff etc.

Although the invention has been explained in relation to its preferred embodiment, many other possible modifications and variations can be made without departing from the spirit and scope of the invention as hereinafter claimed.

1. A safety catheter comprising:
a barrel having
a closed end;
an open end;
a hole formed in the closed end; and
multiple clipper springs connected in the closed end and arranged around the hole;
a cylindrical stand mounted slidably in the barrel and having
a proximal end;
a distal end;
an axis;
an outer surface;
a head formed on and extending longitudinally outward from the distal end;
a channel formed along the axis;
a needle having a sharp end and a blunt end and the blunt end inserted into the channel and the sharp end protruding from the proximal end; and
multiple annular notches formed in and extending radially inward from the outer surface;
a spring mounted slidably in the barrel and around the stand and the needle and having
a proximal end abutting the closed end of the barrel; and
a distal end abutting the head of the stand;
a hub mounted with the barrel and having
a proximal open end;
a distal closed end;
a chamber formed in the proximal open end;
a center channel formed longitudinally and extending in the distal closed end and communicating with the chamber, wherein the clipper springs are compressed in the chamber and let the hub connect with the barrel; and
a cannula connected with the distal closed end and communicating with the channel; and
a seal mounted in the open end of the barrel.

2. The safety catheter as claimed in claim 1 further comprising a cap selectively covering the hub.

3. The safety catheter as claimed in claim 2, wherein the clipper springs are formed in pairs and each pair resembles a pair of tongs.
